(19) United States
(12) Patent Application Publication
(10) Pub. No.: US 2026/0348947 A1
(43) Pub. Date: Nov. 5, 2026

(54) SYSTEMS AND METHODS FOR INCREASING COMPATIBILITY OF DETECTION MATERIALS

(71) Applicant: Morphix Technologies, Inc., Virginia Beach, VA (US)

(72) Inventor: Edward Locke, Norfolk, VA (US)

(21) Appl. No.: 19/196,496

(22) Filed: May 1, 2025

(51) Int. Cl. G01N 33/52

(52) U.S. Cl. CPC G01N 33/52

(57) ABSTRACT

Detection materials comprising a cloaking microparticle are provided. Methods of forming detection materials are also provided. The method comprises the steps of dispersing at least one sensing material and at least one particle material in a solvent and removing the solvent to form the detection material.

### STATEMENT REGARDING FEDERALLY SPONSORED RESEARCH OR DEVELOPMENT

This invention was made with Government support under Contract #FA807518D0017 awarded by the Department of Defense—Defense Technical Information Center. The Government has certain rights in the invention.

### BACKGROUND

This disclosure relates generally to detecting substances and other stimuli and, more particularly, to detection materials and methods of making the same.

One particular use of detection materials is to detect hazardous substances. Throughout the world, at least some individuals may be at risk of exposure to hazardous substances. For example, such individuals may include factory workers, military personnel and first responders. Accordingly, it is desirable to provide individuals at risk with a detection device equipped with sensing materials capable of detecting the presence of hazardous substances. Known detection devices include colorimetric badges, colorimetric tubes, and electrochemical monitors. Detection devices are also used to detect non-hazardous chemicals, pressure, temperature, enzymes, physical abrasion, and electromagnetic radiation, amongst other applications.

However, one particular drawback to these known detection devices is the wide-ranging properties, such as solubility and reactivity, of the various sensing materials incorporated into the detection device. In particular, sensing materials must not only be compatible with other sensing materials for accurate analyte detection device, but also must remain inert to and/or compatible with the substrate materials and other materials such as solvents used for the production of the detection device. Specific substrate materials, such as paper pulp, polymeric films and polymeric gels, are selectively combined with sensing materials to insure compatibility of the various properties of both the sensing materials and substrate materials. In addition, both the sensing materials and the substrate materials must be compatible with the methods and materials used to produce the detection devices and the environment in which the detection device is employed.

The combination of various sensing materials with a substrate material to produce a detection material that incorporates the wide-ranging properties of sensing materials could be used for detecting a plurality of substances or other stimuli. More specifically, it would be desirable to combine currently incompatible sensing materials with each other, with a substrate material, and/or with other materials to produce functional detection materials, as this would facilitate providing both new methods of production as well as new detection materials and the capacity to apply these materials in sensing. Furthermore, a detection material capable of withstanding a plurality of environmental conditions would increase the number of potential users and the number of potential situations in which the sensing materials could be used.

The field of microparticle encapsulation has established robust approaches for producing drug delivery systems which incorporate active pharmaceutical ingredients into polymeric microparticles that provide protection from environmental factors such as humidity, temperature, pH, oxygen, and enzymes as well as provide methods of controlled release. The microparticles can be classified based on their internal structure as microspheres or microcapsules. Microspheres contain drugs homogeneously dispersed within the matrix either suspended or dissolved whereas microcapsules are heterogeneous systems in which the drugs are entrapped within a membranous shell. Critically, these microparticle systems provide a solution to the challenging properties of the active drugs such as poor bioavailability and short half-lives.

The essential properties of microparticle systems provide approaches for broadening the production and use of detection materials. In particular, by combining sensing materials with microparticles the resulting cloaking microparticles can mask or cloak the incompatible properties of the sensing material such as solubility and reactivity. Furthermore, microparticle systems provide a means to combine multiple sensing materials that are otherwise incompatible with a variety of substrate materials and/or process chemicals used in the production of detection materials.

### BRIEF DESCRIPTION

The present disclosure provides detection materials comprising at least one cloaking microparticle, wherein the cloaking microparticle comprises at least one sensing material and at least one particle material and further wherein the cloaking microparticle is less soluble and/or less reactive than the at least one sensing material. When used herein, “less soluble” includes insoluble, and “less reactive” includes non-reactive.

The present disclosure also provides methods of forming a detection material comprising the steps of: forming a dispersion comprising at least one sensing material, at least one particle material, and at least one solvent; and removing the solvent to form the detection material.

### BRIEF DESCRIPTION OF THE DRAWINGS

FIG. 1 shows microscope images at different magnifications of cloaking microparticles in accordance with the embodiments described herein (avg. diameter~200 μm).

FIG. 2 shows the detection of VX agent using paper fiber prepared with a cloaking microparticle material according to the embodiments disclosed herein.

FIG. 3 shows steps of an example method for producing cloaking microparticles as well as detection materials in accordance with the embodiments described herein.

### DETAILED DESCRIPTION

The present disclosure provides detection materials including at least one cloaking microparticle. For typical embodiments, the cloaking microparticles enable detection by incorporating a sensing material into a particle material. In some embodiments, there is a substrate material which immobilizes the cloaking microparticles and provides a medium for the cloaking microparticles to come in contact with analytes of interest. In some embodiments, the detection material is a chemical detection material. As used herein, a chemical detection material refers to a chemically reactive material operable to detect one or more chemicals.

In some embodiments, the at least one cloaking microparticle includes at least one sensing material and at least one particle material. For typical embodiments, the sensing materials encompass indicator chemistry such as acid-base chemistry and redox chemistry. For typical embodiments, the particle material encapsulates the sensing material and provides a medium for the sensing material to be cloaked. In addition, for typical embodiments, the properties of the particle material, such as solubility and reactivity, provide an avenue for “cloaking” the properties of the sensing material.

As used herein, the term “cloaked”, or “cloaking” means a process which changes how a sensing material interacts with other chemicals or the environment. A “cloaked” sensing material has the property of hiding or masking the reactivity or solubility characteristics of the sensing material from other chemicals or the environment preventing unwanted reactivity and/or dissolution. For example, a cloaking microparticle of the current disclosure may comprise a sensing material and a particle material wherein the solubility properties of the cloaking microparticle are more favorable than the solubility properties of just the sensing material.

For purposes of the present disclosure, the sensing material includes both response mechanism materials and response manifestation materials. Specific response mechanism sensing materials include but are not limited to chemically reactive materials, electrochemically sensitive materials, pressure sensitive materials, thermally sensitive materials, enzymatic sensitive materials, physical abrasion sensitive materials, and electromagnetic radiation sensitive materials including X-ray, gamma ray, beta particles, ultraviolet, visible, infrared, microwave, and radio. Specific response manifestation materials include but are not limited to colorimetric materials, fluorescence materials, absorbance materials outside the visible spectrum, electrochemical materials as measured by electrical properties such as conductivity and/or impedance spectroscopy, and thermal materials as measured by calorimetry. In some embodiments, the sensing material is a response mechanism material. In some embodiments, the sensing material is a response manifestation material. In other embodiments, the sensing material is a combination of both response mechanism materials and response manifestation materials.

In some embodiments, the sensing material is a chemically reactive material. In some embodiments, the sensing material is an electrochemically sensitive material. In some embodiments, the sensing material is a pressure sensitive material. In some embodiments, the sensing material is a thermally sensitive material. In some embodiments, the sensing material is an electromagnetic radiation sensitive material. In some embodiments, the sensing material is an enzyme sensitive material. In some embodiments, the sensing material is a physical abrasion sensitive material.

In some embodiments, the sensing material is a colorimetric material. In some embodiments, the sensing material is a fluorescence material. In some embodiments, the sensing material is an absorbance material outside the visible spectrum. In some embodiments, the sensing material is an electrochemical material as measured by electrical properties such as conductivity and/or impedance spectroscopy. In some embodiments, the sensing material is a thermal material as measured by calorimetry.

In an embodiment of the present disclosure, the sensing material may react with substance(s) to exhibit a color change or other sensing change, but not be limited thereto. The sensing material may be a dye, which can be dissolved in water, a polar solvent, or a nonpolar solvent. As to the sensing material, any conventionally known sensing material may be used without limitation, and the sensing material may include, for example, a dye selected from the group consisting of bromophenol blue, bromocresol green, chlorophenol red, bromocresol purple, alizarin red S, alizarin yellow GG, alizarin yellow R, brilliant yellow, bromothymol blue, Congo red, dimethyl yellow, metacresol purple, methyl red, methyl green, methyl orange, phenolphthalein, Clayton yellow, cresol red, crystal violet, erythrosine, malachite green, metanil yellow, neutral red, phenol red, para-methyl red, para-nitrophenol, quinaldine red, thymolphthalein, thymol blue, resazurin, lead acetate, ethyl violet, pentamethoxy red, 2,6-dichloroindophenol, and combinations thereof, but not be limited thereto. Examples of suitable redox sensing materials include dipyridines, benzidines, diphenylene diamine, or metallo-porphyrins, and combinations thereof, but not be limited thereto.

In some embodiments, the particle material is PLA (polylactic acid). In some embodiments, the particle material is PGA (polyglycolic acid). In some embodiments, the particle material is PLGA (poly-(lactic-acid co-glycolic acid)). In some embodiments, the particle material is PCL (polycaprolactone). In some embodiments, the particle material is any of the various cellulose derivatives (ethyl cellulose, hydroxypropyl cellulose, etc.).

In an embodiment of the present disclosure, the particle material can range in size and porosity to effectively cloak the sensing material. In some embodiments the cloaking microparticle size is about 1 nm to about 1 mm.

In some embodiments, the cloaking microparticle is less soluble than the sensing material.

In some embodiments, the cloaking microparticle is less reactive than the sensing material to production, storage, use and/or an environmental condition. In some embodiments, the production, storage, use or environmental condition is selected from the group consisting of water immersion, temperature, humidity, pressure, light, process chemicals, ambient chemicals, or any combination thereof.

In some embodiments, the cloaking microparticle is less reactive than the sensing material to other chemicals in the detection material.

In some embodiments, the cloaking microparticle is more selective than the sensing material to a targeted analyte or set of analytes.

In some embodiments, the substrate material is a paper pulp. In some embodiments, the substrate material is a polymer. In some embodiments, the substrate material is a gel. In some embodiments, the substrate material is an emulsion. In some embodiments, the substrate material is glass fibers. In some embodiments, the substrate material is a combination of materials. In some embodiments, the substrate material is a gas (e.g. air).

The present disclosure also provides detection materials according to any of the previous embodiments further including a plurality of cloaking microparticles.

The present disclosure also provided methods of forming a detection material including the steps of dispersing at least one sensing material and at least one particle material in at least one solvent and removing the solvent.

The present disclosure also provides methods of forming a detection material including the steps of combining at least one sensing material with at least one particle material.

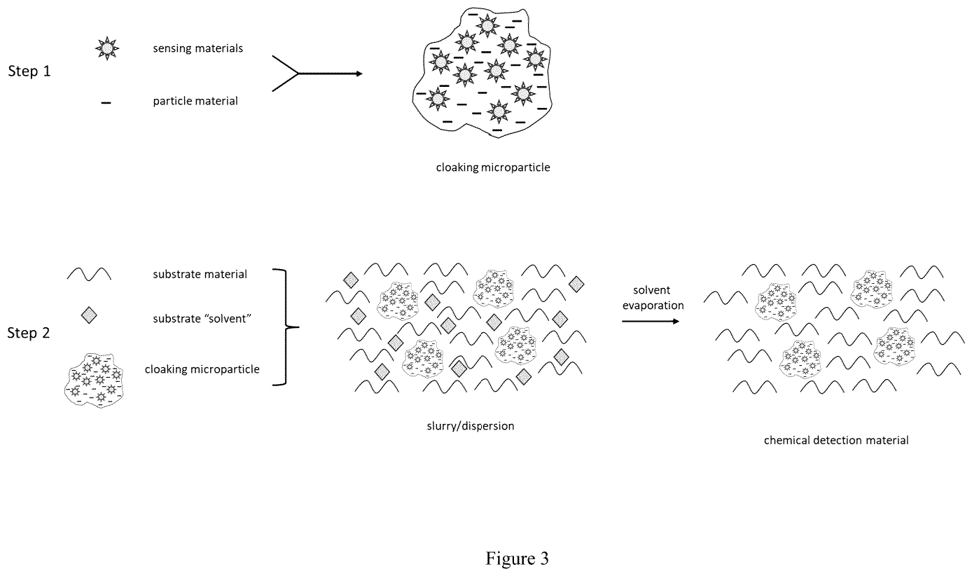

The present disclosure also provides methods of forming a detection material including the steps of dispersing at least one cloaking microparticle and at least one substate material in at least one solvent and removing the solvent. In typical embodiments, the method involves a first step of combining a sensing material and a particle material as depicted in step 1 of FIG. 3. The method then proceeds to step 2 wherein the cloaking microparticle produced in step 1 is combined with a substrate material in the presence of a solvent to produce a slurry/dispersion and upon removal of the solvent a detection material is obtained as depicted in step 2 of FIG. 3.

The present disclosure also provides methods of forming a detection material including the steps of dispersing at least one cloaking microparticle and at least one substate material and obtaining the detection material.

The present disclosure also provides methods of forming a detection material including the steps of dispersing and/or emulsifying at least one sensing material and at least one particle material and 3-D printing or otherwise extruding the resulting cloaking microparticle to obtain the detection material.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: Preparation of a Detection Material With a Water-Soluble Sensing Material, a Water-Insoluble Particle Material, and a Water-Based Substrate Material

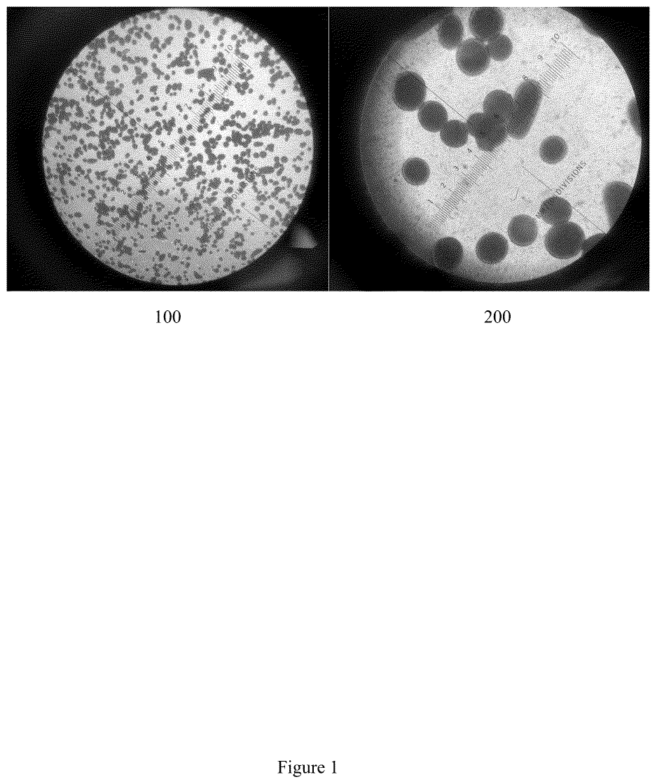

Bromocresol purple indicator (0.1-15 mg/mL) is dissolved in a solution of ethyl cellulose (EC, 48% ethoxylated, viscosity 4 cPs) in ethanol (4 g EC in 100 mL ethanol). The solution is then transferred to a stirring solution of water and the resulting cloaking microparticle is extracted by a method such as filtration or centrifugation. FIG. 1 shows a first microscope image 100 and a second microscope image 200. Microscope images 100 and 200 are taken at different magnifications and show the resulting cloaking microparticles. The cloaking microparticle is then dispersed in a solution of a water-based polymer, glass fiber and/or pulp forming a detection material slurry/dispersion that is then film casted or sheet-formed to obtain a detection material.

### Example 2: Preparation of a Detection Material With an Organic-Soluble Sensing Material, an Organic-Insoluble Particle Material, and an Organic-Based Substrate Material

Ethyl orange sodium salt indicator (0.1-5 mg/mL) is dissolved in a solution of polyvinyl alcohol (PVA, 88-90% hydrolyzed) in water (3 g PVA in 100 mL water). The solution is transferred to a stirring solution of methanol and the resulting cloaking microparticle is extracted by a method such as filtration or centrifugation. The cloaking microparticle is then dispersed in a solution of an organic-based polymer forming a detection material slurry/dispersion that is then film casted or sheet-formed to obtain a detection material.

### Example 3: Solventless Preparation of a Detection Material

A sensing material is added to a thermoset precursor particle material and emulsified to provide a cloaking microparticle. The cloaking microparticle is then 3-D printed or otherwise extruded to obtain a detection material.

### Example 4: Detection of VX With a Detection Material Comprising a Cloaking Microparticle

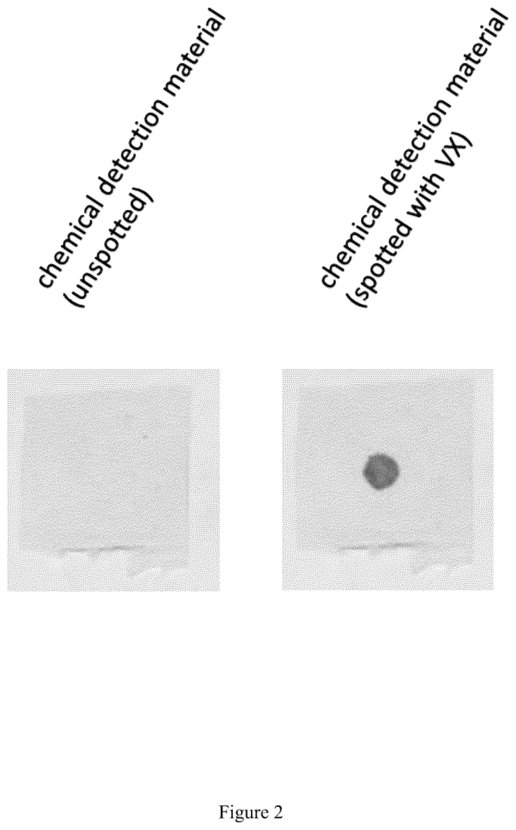

FIG. 2 shows the detection material prepared according to example 1 before treatment (left) and after treatment (right) with VX agent.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

The terminology used herein is for the purpose of describing particular embodiments only and is not intended to be limiting.

What is claimed is:

1. A detection material comprising at least one cloaking microparticle, wherein
a. the cloaking microparticle comprises at least one sensing material and at least one particle material; and
b. the cloaking microparticle is less soluble and/or less reactive than the at least one sensing material.

2. The detection material according to claim 1, wherein the sensing material is at least one of a response mechanism material and/or a response manifestation material.

3. The detection material according to claim 1, wherein the sensing material is a chemically reactive material.

4. The detection material according to claim 1, wherein the particle material is selected from the group consisting of synthetic polymers, non-synthetic polymers, or any combination thereof.

5. The detection material according to claim 1, wherein the cloaking microparticle is less soluble than the at least one sensing material under production, storage, use, and/or environmental conditions.

6. The detection material according to claim 5, wherein the production, storage, use, or environmental condition is selected from the group consisting of water immersion, temperature, humidity, pressure, light, process chemicals, ambient chemicals, or any combination thereof.

7. The detection material according to claim 1, wherein the cloaking microparticle is less reactive than the at least one sensing material under production, storage, use, and/or environmental conditions.

8. The detection material according to claim 7, wherein the production, storage, use, or environmental condition is selected from the group consisting of water immersion, temperature, humidity, pressure, light, process chemicals, ambient chemicals, or any combination thereof.

9. The detection material according to claim 1, wherein the cloaking microparticle is less reactive than the at least one sensing material to other chemicals in the detection material.

10. The detection material according to claim 1, wherein the cloaking microparticle is selective for a targeted analyte or a set of analytes.

11. The detection material according to claim 1, further comprising at least one substrate material.

12. The detection material according to claim 11, wherein the at least one substrate material is selected from the group consisting of a paper, a paper pulp, a polymer, a gel, an emulsion, glass, a glass fiber, a gas, and combinations thereof.

13. A method forming a detection material comprising the steps of:
a) forming a dispersion comprising at least one sensing material, at least one particle material, and at least one solvent; and
b) removing the solvent to form the detection material.

14. The method according to claim 13, further comprising at least one substrate material.

15. The method according to claim 14, wherein the at least one substrate material is selected from the group consisting of paper, a paper pulp, a polymer, a gel, an emulsion, glass, a glass fiber, a gas, and any combinations thereof.

16. The method according to claim 13, wherein the sensing material is at least one of a response mechanism material and/or a response manifestation material.

17. The method according to claim 13, wherein the sensing material is a chemically reactive material.

18. The method according to claim 13, wherein the at least one particle material is selected from the group consisting of synthetic polymers, non-synthetic polymers, or any combination thereof.

19. The method according to claim 13, wherein the detection material is less soluble than the at least one sensing material under production, storage, use, and/or environmental conditions.

20. The method according to claim 19, wherein the production, storage, use, or environmental condition is selected from the group consisting of water immersion, temperature, humidity, pressure, light, process chemicals, ambient chemicals, or any combination thereof.

21. The method according to claim 13, wherein the detection material is less reactive than the at least one sensing material under production, storage, use, and/or environmental conditions.

22. The method according to claim 21, wherein the production, storage, use, or environmental condition is selected from the group consisting of water immersion, temperature, humidity, pressure, light, process chemicals, ambient chemicals, or any combination thereof.

23. The method according to claim 13, wherein the detection material is selective for a targeted analyte or a set of analytes.

24. The method according to claim 13, wherein the at least one solvent is selected from the group consisting of polar, non-polar, or any combinations thereof.
